(19) United States
(12) Patent Application Publication
(10) Pub. No.: US 2024/0024685 A1
(43) Pub. Date: Jan. 25, 2024

(54) Implantable Platforms For Transcranial And Long-Range Optogenetics

(71) Applicant: Arizona Board of Regents, Tucson, AZ (US)

(72) Inventor: Philipp GUTRUF, Tucson, AZ (US)

(73) Assignee: Arizona Board of Regents, Tucson, AZ (US)

(21) Appl. No.: 18/225,480

(22) Filed: Jul. 24, 2023

Related U.S. Application Data
(60) Provisional application No. 63/391,364, filed on Jul. 22, 2022.

(51) Int. Cl. A61N 1/36; A61N 1/40

(52) U.S. Cl. CPC A61N 1/36171; A61N 1/40

(57) ABSTRACT

The present disclosure provides an implantable optogenetic stimulation device. In one embodiment the device includes a housing and an optoelectronic stimulation circuit for light delivery for optogenetic stimulation. The stimulation circuit includes energy harvesting circuitry to receive radio frequency (RF) energy; one or more capacitor storage elements to store energy associated with the RF energy; a light emitting diode (LED) to generate a light source for optogenetic stimulation at a selected frequency and duty cycle; and controller circuitry to discharge the one or more capacitor storage elements at a selected duty cycle to cause the LED to generate pulsed light at the selected duty cycle with energy requirement above the peak power capability of the RF harvesting circuit.

This application claims the benefit of U.S. Provisional Application Ser. No. 63/391,364, filed July 22, which is hereby incorporated by reference in its entirety.

### TECHNICAL FIELD

The present disclosure relates to implantable platforms for transcranial and long-range optogenetics.

### BACKGROUND

Wireless, battery-free, and fully subdermally implantable optogenetic tools are poised to transform biological research in freely behaving animals. Current devices are sufficiently small, thin, and light for subdermal implantation, reducing the impact compared to tethered methods. Yet, current limitations in wireless power delivery require invasive modes of stimulus delivery that penetrate the skull and disrupt the blood brain barrier, causing tissue displacement, neuronal damage, and scarring. Power delivery constraints also sharply curtail operational area size.

The ability to optogenetically activate select neuronal populations enables a vast array of experiments ranging from basic exploratory research to therapeutic applications. Current technology for light stimulus delivery is constrained by one of more of the following—tethers, externalized head stages, or bulky battery powered solutions, which render some classes of experiments and applications problematic and others impossible. These invasive methods require implanting devices into the area of interest, causing tissue damage.

The recent emergence of wireless, battery-free, and subdermally implantable optogenetic activation devices dramatically expands experimental capabilities over conventional tethered or externalized head-mounted solutions. Critical advantages include minimized impact on subject, device operation in multiple subjects or locations at the same time, and multimodal operation that combines optogenetic activation with, for example, fluid delivery. Collectively, these advances provide a quantitative expansion of optogenetic and cell specific recording tools over tethered and battery powered technologies and broaden the scope of experimental paradigms that can be explored in small vertebrate models.

However, remaining fundamental limitations of wireless and battery-free systems relate to the nature of power delivery, which in most cases relies on magnetic resonant coupling, mid field, or far field power delivery. All of these approaches suffer from power drop-offs as the device moves away from the power transmitting antenna. Existing power delivery solutions limit the use of these devices to −13,500 cm³ for continuous operation. Given this constraint, high powered applications such as optogenetic activation through the skull or long-range operation cannot be achieved with the power harvesting capabilities of subdermally implanted devices. A realization of these capabilities would eliminate barriers in the design of experimental paradigms or use cases. This could enable diverse experiments that either leverage ethologically grounded, naturalistic environments that include burrows and obstacles for rodents, or those that take advantage of scale, for instance allowing the exploration of neural mechanisms underlying the formation of hippocampal and entorhinal cortex “place” and “grid” cells in large, ethologically relevant environments. The expansion of the 3D environment size optimizes experiments for 3D mobile birds and bats, also opening the door for innovative experiments in primates, which leverage the recent developments in genetic traction for these models. A separate, likewise potentially transformative benefit of the current advances involves increased delivered power for transcranial access. This application is optimal for longer wavelength sensitive opsins, due to superior tissue penetration by longer wavelength light. Transcranial activation of long wavelength sensitive opsins and halorhodopsins (e.g., ChrimsonR [590 nm], ReaChR [590 nm], C1V1 [590 nm], Arch [566 nm], NpHR [580 nm] and ChRmine [635 nm]) can eliminate the negative impact of penetrative probes on the brain paving the way for improved, less invasive neuroscience.

### BRIEF DESCRIPTION OF THE DRAWINGS

Features and advantages of various embodiments of the claimed subject matter will become apparent as the following Detailed Description proceeds, and upon reference to the Drawings, wherein like numerals designate like parts, and in which:

FIG. 1 illustrates an overview and operational characteristics of a wireless, subdermally implantable device with capacitive energy storage according to several embodiments of the present disclosure; where FIG. 1A illustrates 3D rendering and photographic image of the device highlighting a miniaturized profile; FIG. 1B illustrates a current output curve of the capacitive energy storage; FIG. 1Ci illustrates a device circuit diagram according to the teachings of the present disclosure; and FIG. 1Cii illustrates voltage measurements of capacitor bank and current measurements of load for high load and low load applications;

FIG. 2 illustrates operation principles and electrical, mechanical and optical characterization of a long-range implantable device according to several embodiments of the present disclosure; where FIG. 2A illustrates average electrical power consumption for a range of duty cycles and irradiances; FIG. 2B illustrates an example circuit diagram; FIG. 2C illustrates an exploded view of one example device according to one embodiment; FIG. 2D illustrates the assembled example device of FIG. 2C balanced on a fingertip; FIG. 2E illustrates an image of device serpentine antenna being stretched with inset showing mechanical strain finite element simulation; FIG. 2F illustrates magnetic finite element simulation of 70 cm×70 cm arena; FIG. 2G illustrates rectification behavior of a long-range device with 6, 8 and 10 turns in the center of a 70×70 cm arena at a height of 3 cm and an input power of 10 W.; FIG. 2H illustrates power distribution of 10 turn device in a 70 cm×70 cm arena with 10 W input power at heights of 3, 6, and 8 cm from arena floor; FIG. 2I illustrates an image of devices with red, orange, and blue μ-ILEDs activated in a 70 cm×70 cm arena; FIG. 2J illustrates IV curves for red, orange, and blue μ-ILEDs; and FIG. 2K illustrates images of devices with red, orange, and blue μ-ILEDs;

FIG. 3 illustrates 3D imaging and impact on behavior according to one example embodiment; where FIG. 3A illustrates combined MRI and CT 3D rendering of the implantable device implanted in mouse; FIG. 3B illustrates a side view CT image of the implantable device; FIG. 3C illustrates device operation in mouse implanted with device in a 50 cm×50 cm cage with 8 W RF input; FIG. 3D illustrates a trajectory map for of control of an animal with the implantable device; FIG. 3E illustrates another trajectory map for of control of an animal with the implantable device; and FIG. 3F illustrates a comparison of exploration rate and distance traveled for control animal and animal implanted with the device;

FIG. 4 illustrates operational principles and characterization of the transcranial implantable device according to one example embodiment; where FIG. 4A illustrates average power required for combinations of duty cycles and optical powers; FIG. 4B illustrates an example circuit for the implantable device capable of high intensity stimulation; FIG. 4C illustrates an exploded view of one example implantable device according to an embodiment; FIG. 4D illustrates an image of the device of FIG. 4C balanced on finger tip; FIG. 4E illustrates an image of strained device with inset of finite element simulation indicating applied and resulting strain; FIG. 4F illustrates electrical and optical power with increasing current for blue and red μ-ILEDs; FIG. 4G illustrates an example schematic used for line measurements of blue and red light irradiance through skull and brain; FIG. 4H illustrates irradiance measurements for mouse skull and brain for red and blue μ-ILEDs; FIG. 4I illustrates a Monte Carlo simulation of light propagation through skull for a red light source with 13 mW optical power; FIG. 4J illustrates thermal impact analysis of red μ-ILED with 13 mW optical power at steady state; and FIG. 4K illustrates experimentally measure brain temperature for an intact skull at increasing brain depth from the site of the μ-ILED with the transcranial stimulation device operated at 10 Hz and a 1% duty cycle for 5 minutes; and

FIG. 5 illustrates an example of transcranial optogenetic stimulation in M2 drives rotational behavior; where FIG. 5A is a CT image of a subdermally implanted device on the skull of a mouse (Scale bar: 5 mm); FIG. 5E is a schematic of virus transduction and experimental timeline; FIG. 5C is a schematic of transcranial optogenetic stimulation in M2 and an example image of ChrimsonR expression (Scale bar: 1 mm); FIG. 5D illustrates example frames across a 9-s long stimulation episode, where clots show the tracked body positions tracked; FIG. 5E illustrates example traces of mice expressing fluorophore or ChrimsonR with or without transcranial optogenetic stimulation (Scale bar: 3 mm); FIG. 5F is a schematic illustrating the calculation of rotation degree for each frame (0.5-s bin); FIG. 5G (left side) is a graph of summary data showing the cumulative rotation degrees in mice expressing ChrimsonR during a 40-s long episode, and FIG. 5G (right side) is a graph of mice expressing control fluorophore; FIG. 5H illustrates summary data showing the total degrees rotated in each experimental condition; FIG. 5I shows example images of c-Fos staining in M2 of mice expressing CrimsonR-tdT with or without transcranial optogenetic stimulation; and FIG. 5J illustrates summary data showing the number of c-Fos neurons in individual mice (Left), the distribution of individual c-Fosparticle intensities (Middle), and cumulative frequency of c-Fos particle intensities (Right).

Although the following Detailed Description will proceed with reference being made to illustrative embodiments, many alternatives, modifications and variations thereof will be apparent to those skilled in the art.

### DETAILED DESCRIPTION

This disclosure provides a digitally managed, highly miniaturized, capacitive power storage to wireless and subdermal implants. This approach enables power delivery to optoelectronic components to enable two classes of new applications: transcranial optogenetic activation up to 5 mm deep into the brain or quadrupling experimental arenas for wireless optogenetics to over 1 m² in size. This methodology significantly increases optogenetic activation capabilities in freely moving subjects and enables previously impossible experiments.

This disclosure also provides a wireless, battery-free, subdermal implant that enables high intensity optogenetic light stimulus through the skull in large experimental arenas. This approach allows eliminating brain tissue damage or quadruples experimental arena size, enabling new ethologically grounded neuroscience experiments. According to the teachings herein, the limitations described above may be overcome by creating the means for harnessing energy continuously, using highly miniaturized capacitive energy storage, and by digitally managing power delivery to optoelectronic components. This level of control enables transcranial optogenetic stimulation and long-range operation in arenas of large volume.

Optogenetic activation is conventionally implemented by introducing pulsed delivery of light. The energy requirement for a device that produces this stimulus is likewise pulsed, defining peak demand. When considering the average power demand, however, energy requirements are typically much lower, because optogenetic activation usually operates with a duty cycle of 10-30%. To harness the energy in between stimulation pulses, an energy storage is required that can retain the energy needed to buffer the delivery of one pulse of light, while storing the energy harvested from the electromagnetic field without extensive losses. In the case of subdermally implantable devices, several additional parameters have to be considered, beyond storage capacity. The size, energy density, electrical characteristics, peripheral components needed for operation, and commercial availability are all important to realize devices of broad impact to the neuroscience community. Based on these considerations, capacitive energy storage emerges as the most suitable technology, because of the relatively high energy density (1.38 J/cm³ for a 0402 form factor 22 μf ceramic capacitor and 0.639 J/cm³ for a 0201 2.2 μf ceramic capacitor, with an operational voltage limit of 5.6 V). Additionally, the capability to operate over a wide voltage range without the need for advanced management components, as well as the capability to deliver its energy rapidly, enables the support of high peak power demands. These ceramic capacitors are also available in small form factors that can be easily integrated into electronic circuits with schemes that enable system level flexibility critical to conform to curvilinear surfaces.

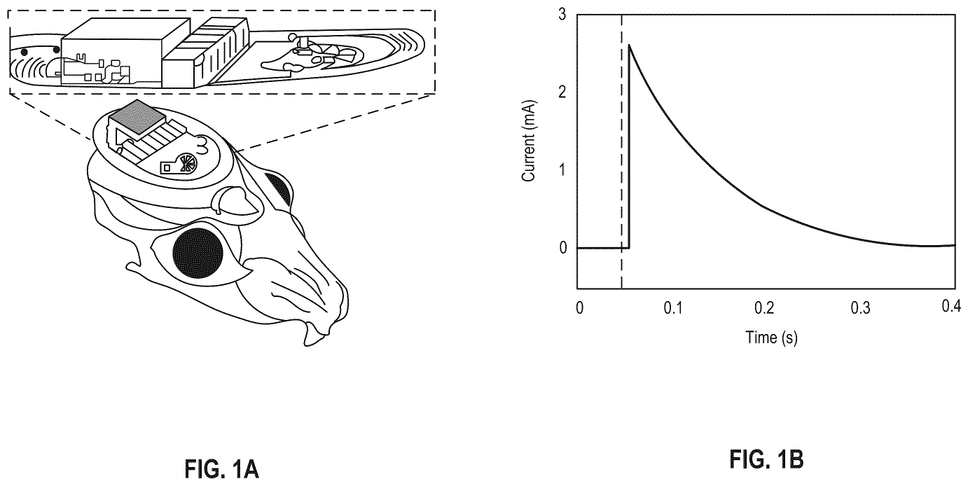

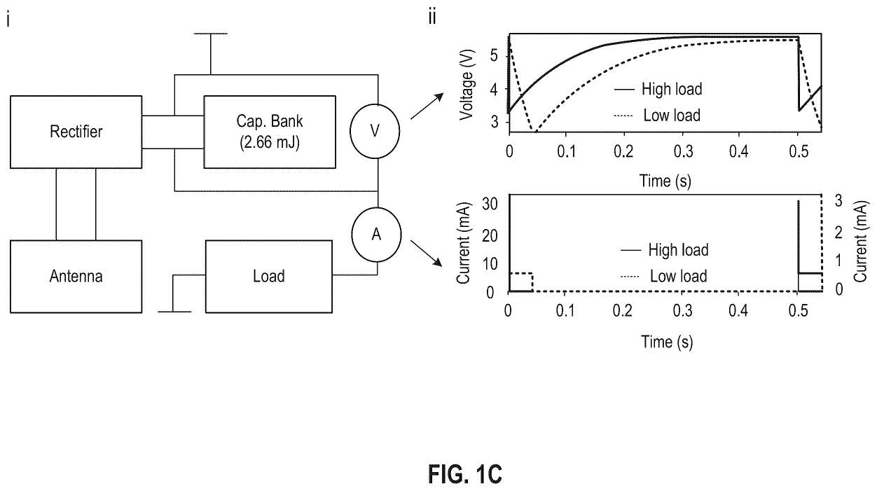

FIG. 1 illustrates an overview and operational characteristics of a wireless, subdermally implantable device with capacitive energy storage according to several embodiments of the present disclosure. A 3D rendering and photographic image of device makeup is illustrated in FIG. 1A, illustrating a minimal footprint and profile that enable subdermal implantation, as shown compared to a mouse cranium. The capacitive energy storage of this device (1.38 J/cm³) and total capacitive energy storage (2.76 mJ) is comprised of eight 22 μF (total of 176 μF) capacitors arranged in a parallel circuit. In some embodiments, the components selected may be commercially available and seamlessly integrate with the flexible circuit of the subdermally implantable device.

Features of the operational scheme are highlighted in FIG. 1B, demonstrating the ability to supply currents of up to 2.6 A when discharged through a 0.5Ω load. This, together with a low self-discharge rate, allows for a flexible delivery of the power stored within the device. FIG. 1C-i shows a simplified electrical circuit diagram of the implantable device with a capacitor bank. In an example embodiment, the capacitor bank stores up to 2.76 mJ of energy when fully charged with a voltage of 5.6V. FIG. 1C-ii illustrates the capacitor bank voltage in two use scenarios which involve a high load application: high powered pulse delivery to optoelectronic components for transcranial stimuli for short periods of time; and a low load application with regular optogenetic stimuli for extended periods of time. Both applications have similar average energy requirements of 0.11 mJ per pulse. Capacitors and the linear dropout regulator (LDO) can handle an unregulated voltage of up to 5.6 V and provide a stable output voltage (3.3 V for high load and 2.7 V for low load applications). The energy stored in the system represented by the voltage which decays as energy is released, enables a utilization of a voltage drop from 5.6 V to 3.3 V (Δ 2.3 V) for high load applications and from 5.6 V to 2.7 V (Δ 2.9 V) for low load applications. This operational voltage range results in 2.1 mJ of usable energy in the capacitor bank for low load applications and 1.8 mJ for high load applications.

Illumination events of the micro-Inorganic Light Emitting Diode (μ-ILED) load are highlighted in FIG. 1C-ii. Here, the two rates of current delivery sourced from the capacitor bank are shown. For high currents, the corresponding voltage at the capacitor bank decays rapidly and can provide high intensities of light for a short pulse. If current demand is reduced, longer stimuli are enabled at the same energy harvesting rate. Following from these operational parameters, two discrete operational modes for use in optogenetic neural activity control are demonstrated in this work, transcranial stimulation, and long-range applications. While the examples provided herein are generally directed to optogenetic stimulation applications, the teachings of the present disclosure may be applied to various battery-free device categories, such as, photometry methods, electrical stimulation for heart defibrillation or deep brain stimulation, and controlled drug delivery schemes.

Expanding Arena Volume for Optogenetics

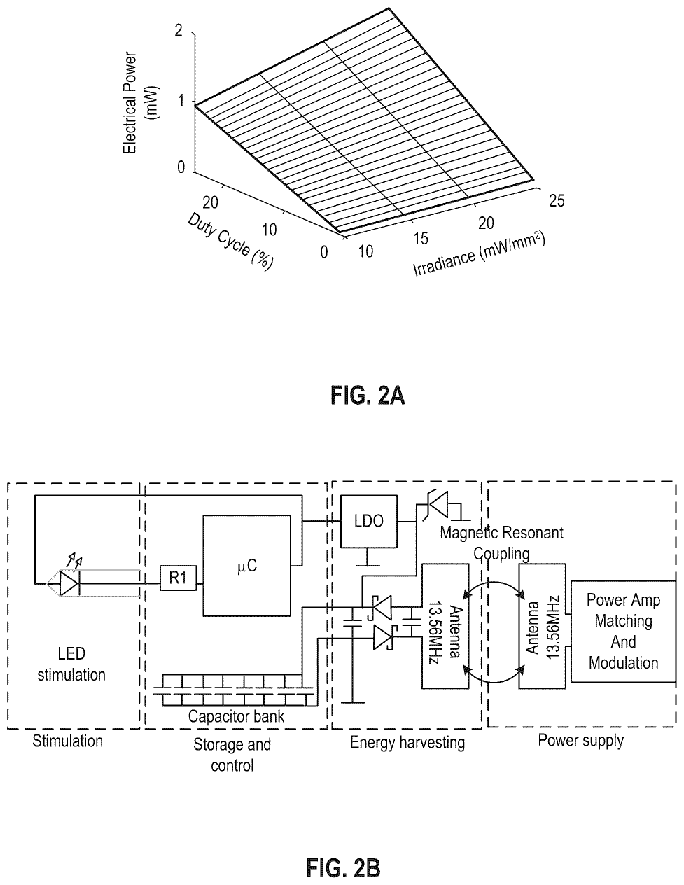

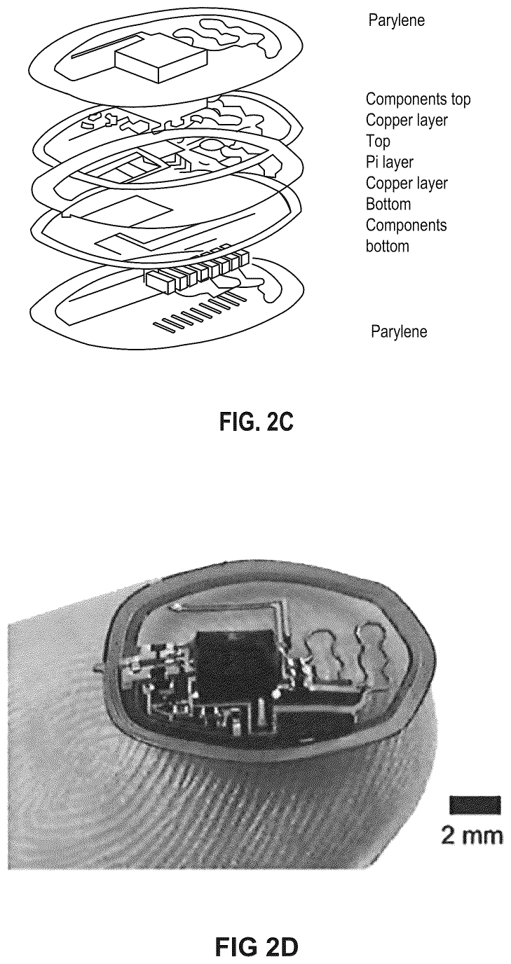

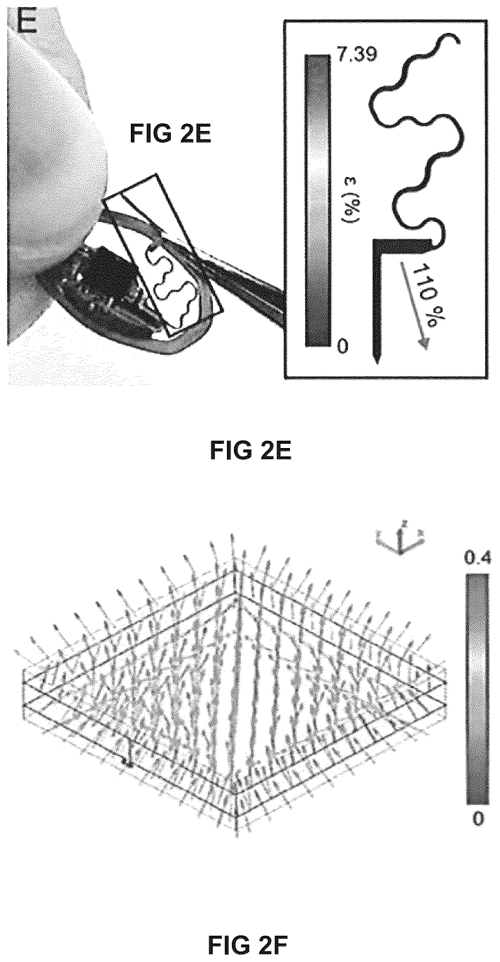

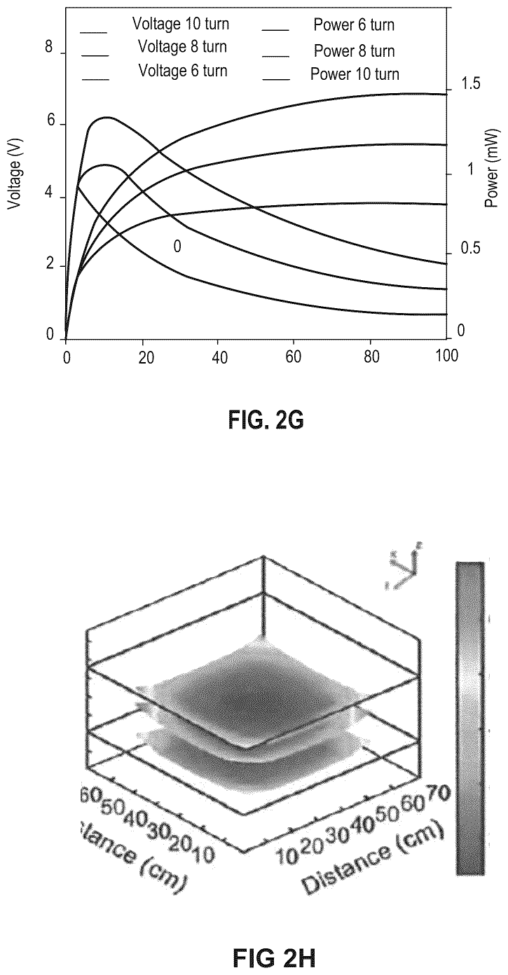

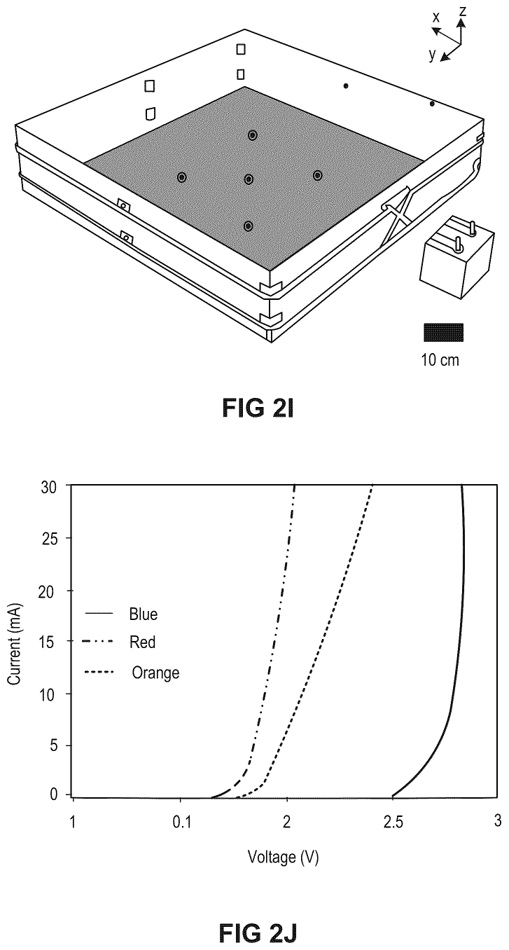

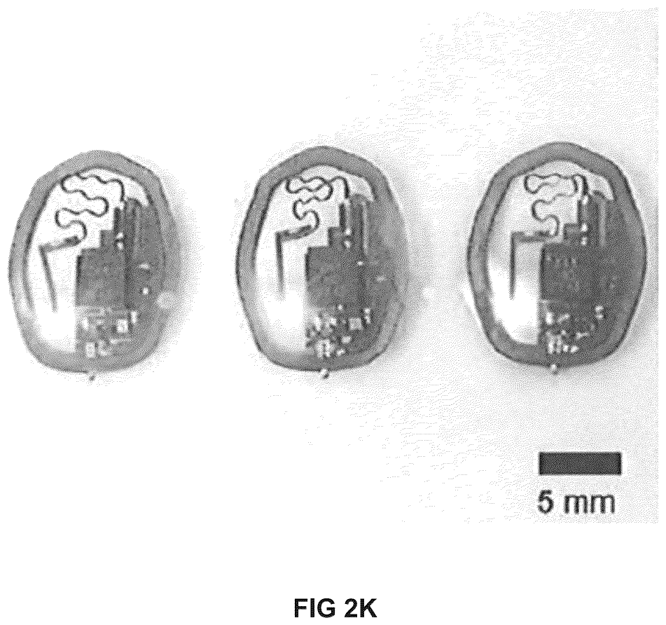

Harnessing the capabilities of the capacitive storage significantly expands arena sizes for wireless battery free optogenetics. FIG. 2 illustrates the electrical, mechanical, and optical design characteristics of devices tailored to work in large arena volumes. Scenarios combining a range of duty cycles and intensities at specific input powers are graphically represented in FIG. 2A. In a typical use scenario, device duty cycles and irradiances of 20% and 10 mW/mm², or duty cycles and irradiances of 10% of 25 mW/mm², represent frequently chosen parameters. These can be guaranteed to operate with as low as 0.75 mW electrical power continuously supplied to the implant.

The energy supplied by the capacitor bank is harnessed by an LDO (2.7 V) to stabilize the operational voltage, and a small outline microcontroller (μC) that controls the μ-ILED in current sink configuration, shown in an electrical schematic in FIG. 2B. Component selection is made with miniaturization as a priority, with example components described below. The result is an 87 mg device with dimensions of 13.50×10.26×0.89 mm and displacement volume of 20.4 mm³.

A layered rendering of the device capable of long-range operation in freely moving subjects is illustrated in FIG. 2C. The capacitor bank for energy storage is located beneath the bottom copper layer to reduce electronics footprint, enabling flexible placement of the injectable light source within the device perimeter. Magnetic resonant coupling (13.56 MHz) between the primary antenna surrounding the experimental arena and the secondary millimeter-scale antenna on the device provides energy wirelessly. Encapsulation is realized with a layer of parylene to create a thin, flexible platform capable of seamless mechanically integration with the curved skull of the subjects.

A photographic image of the device in FIG. 2D highlights the thin and flexible form factor that facilitates subdermal implantation in rodents. The serpentine structure connecting the main body with the probe is designed to stretch extensively during the surgical procedure. The photographic image in FIG. 2E demonstrates this capability, and the inset displays the result of a finite element simulation, subjecting the serpentine to 110% strain (5.53 mm displacement). In this example, the copper layer experiences a maximum strain of 3.58%, within the elastic regime, enabling repeated strain cycles of the serpentine without loss in conductivity. This robust strain performance allows for facile placement of the probe within the perimeter of the device to target various brain regions as well as the periphery, as well as enabling normal animal movement without device breakdown.

Primary antenna performance is critical for operation in large experimental arenas. To cover large volumes and surface areas, the primary antenna is configured in a dual loop arrangement (4 and 11 cm loop from cage floor) over an arena of 70×70 cm. FIG. 2F illustrates a finite element simulation of the magnetic field (details of the simulation are described below in the Methods section). The resulting magnetic field strength is symmetric throughout the cage with a slight reduction near the tuner and loop crossover, with elevated magnetic field density in the corners. This reflects comparable field distribution to smaller enclosures. It is noted that larger antennas become difficult to properly tune at 13.56 MHz operational frequency because the self-resonance approaches the working frequency of the system and effectively limits cage sizes to 70×70 cm with a dual loop antenna setup.

Power harvesting of the secondary antenna, the antenna on the implant, is maximized by optimizing the number of turns in the coil. Power harvesting behavior of devices with equivalent trace spacing (100-μm-wide traces with 50 μm spacing) with 6, 8, and 10 turns are compared by incrementally increasing device load shown in FIG. 2G. Harvesting capabilities of 1.34 mW are obtained with a 10 turn antenna device at a voltage of 3.67 V and a load of 10 kΩ in the center of a 70×70 cm arena, at a height of 3 cm from the arena floor (10 W RF input power). These parameters match the device's operating voltage during capacitor bank recharge events. The corresponding measurements of power distribution throughout the large cage with a 10 turn device and an RF input of 10 W is shown in FIG. 2H at physiologically relevant heights of 3, 6 and 8 cm. Antenna performance is optimized for large enclosures. Following this optimization, sufficient energy to operate devices at the duty cycle and intensity combinations highlighted in FIG. 2A is available for the implant, enabling experimental paradigms that utilize the whole volume of the experimental arena. Operational area and volume are 4900 cm² and 68600 cm³, respectively, reflecting a 444% increase in surface area and 408% increase in volume over prior work. Device operation in even larger arenas (9,400 cm²) can be achieved by combining multiple antennas and RF power sources. In a proof of principle experiment we demonstrate outfitting the floor of an experimental arena with two single loop antennas (65×65 cm) with 15 cm spacing to result in a coverage of 145×65 cm. The distance between the antennas is chosen to be sufficiently far to minimize resonant interaction, but close enough for the near field to extend outside the primary antenna loop. This design allows powering devices located between the antennas. Minimum power in this experimental arena is 0.59 mW, enabling the operation of standard (invasive) optogenetic activation protocols in previously impossible arena sizes for untethered animals.

The capability to supply a range of system voltages enables the utilization of various μ-ILED's that feature turn-on voltages of 1.8 to 2.6 V and thus enables the use with a multitude of opsins and halorhodopsins. A photographic image of multiple devices powered simultaneously with a range of activation wavelengths operating in a 70×70 cm enclosure is shown in FIG. 2I. IV curves for blue, red, and orange μ-ILED's and corresponding images of devices equipped with the optoelectronic components are shown in FIGS. 2J and 2K, respectively.

In Vivo Characterization of Implanted Devices

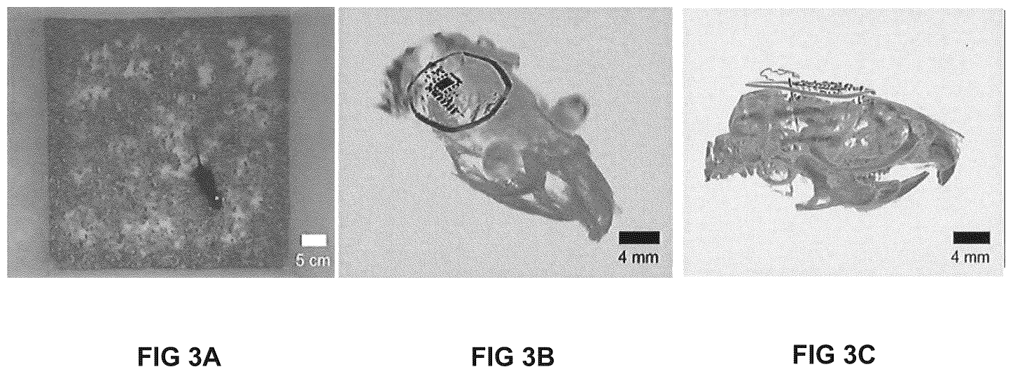

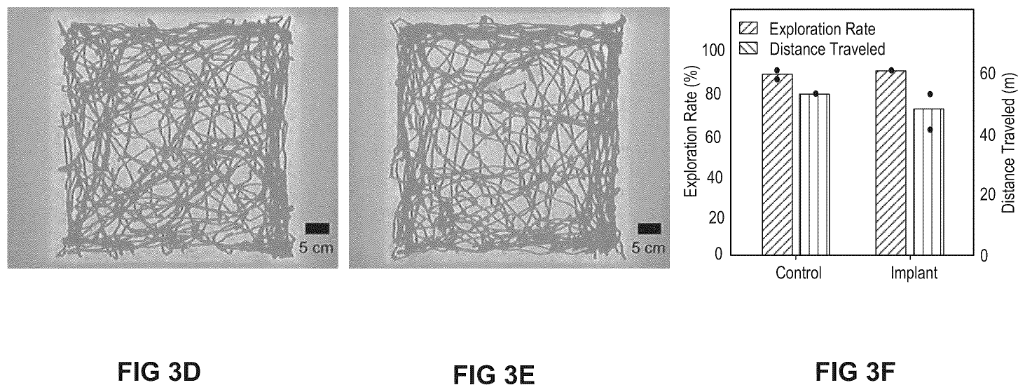

An additional advantage of utilizing ceramic capacitors as an energy storage for devices is minimal impact on 3D imaging, enabling the use of magnetic resonant imaging (MRI) and computed tomography (CT). This selection overcomes imaging difficulties associated with devices that feature magnetic components. The combined 3D rendering of an MRI and CT image is shown in FIG. 3a, highlighting the compatibility with these technologies. The side view shown in FIG. 3b highlights the small footprint, thin makeup, and soft mechanics of the device that enable facile subdermal implantation on the skull and ensure fast recovery of the subject. Device operation is verified visually in a typical open field experimental arena (50 cm×50 cm×40 cm) for mice.

FIG. 3A shows a photographic image with a subject during an open field behavioral assay with active optogenetic stimulation (dual loop antenna, 8 W RF input). Performance of the device is consistent throughout the assay, showing a freely moving subject during active stimulation with a probe subdermally implanted facing toward the epidermis for visual confirmation of device function. An additional advantage of utilizing ceramic capacitors as an energy storage for devices is the minimal impact on 3D imaging, enabling the use of MRI and computed tomography (CT). This overcomes imaging challenges for devices that contain magnetic components. The combined 3D rendering of an MRI and CT image is shown in FIG. 3B. The side view shown in FIG. 3C highlights the small footprint, thin makeup, and soft mechanics of the device that enable facile subdermal implantation on the skull and ensure fast recovery.

To evaluate whether locomotor behavior is altered by the presence of a subdermally implanted device, in the absence of stimulation or opsin expression, a distance traveled in an open field arena for several mice may be measured. Example trajectory maps for a control animal and an animal with an implant are shown in FIGS. 3D and 3E, respectively. The total distance traveled and exploration rates did not vary between mice with and with-out an inactive implant (as illustrated in the graph of FIG. 3F), suggesting that the subdermal implant has a minimal impact on basic aspects of locomotion, unlike external cables used in wired optogenetics.

Transcranial Optogenetic Stimulation

The capability of the capacitive energy storage to deliver high currents in short pulses, as shown in FIGS. 1B and 2B, enables applications where high intensity light pulses are required. Transcranial stimulation has previously been realized in anesthetized animals with high intensity light pulses that penetrate the skull without invasive procedures. Due to technological limitations, namely low current delivery capabilities, low voltage, and the relatively large form of currently available miniaturized batteries, this approach has not been demonstrated wirelessly in freely moving subjects.

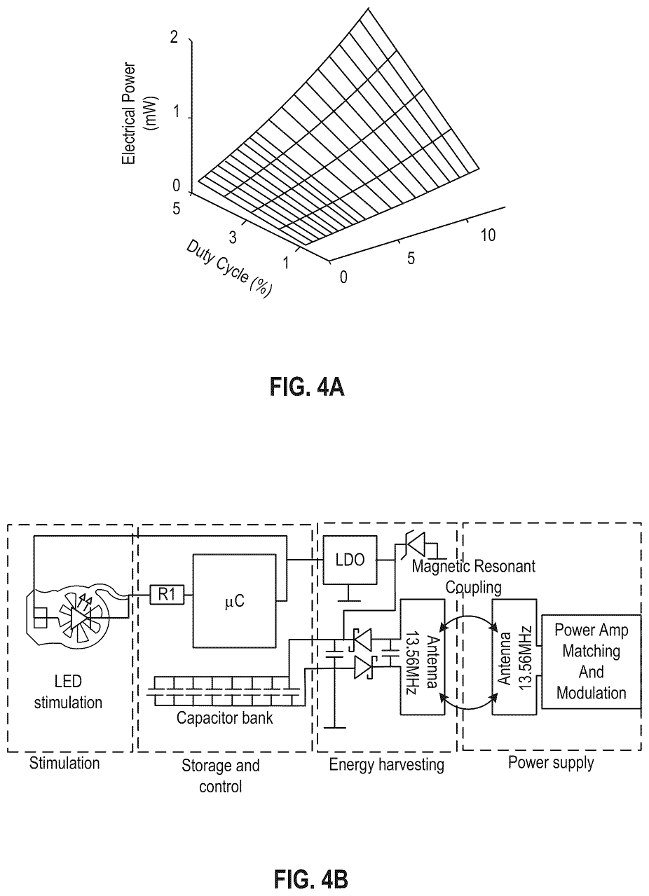

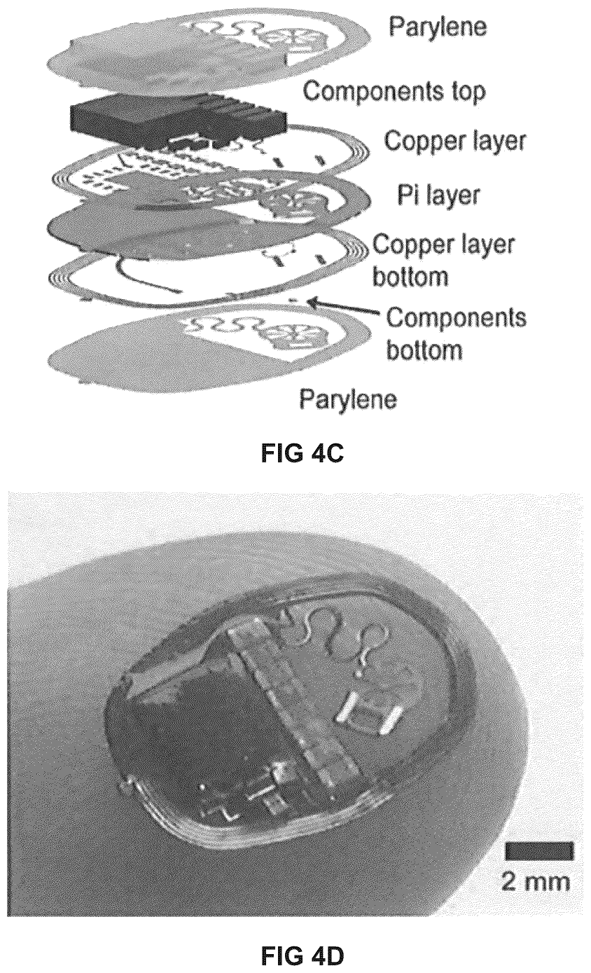

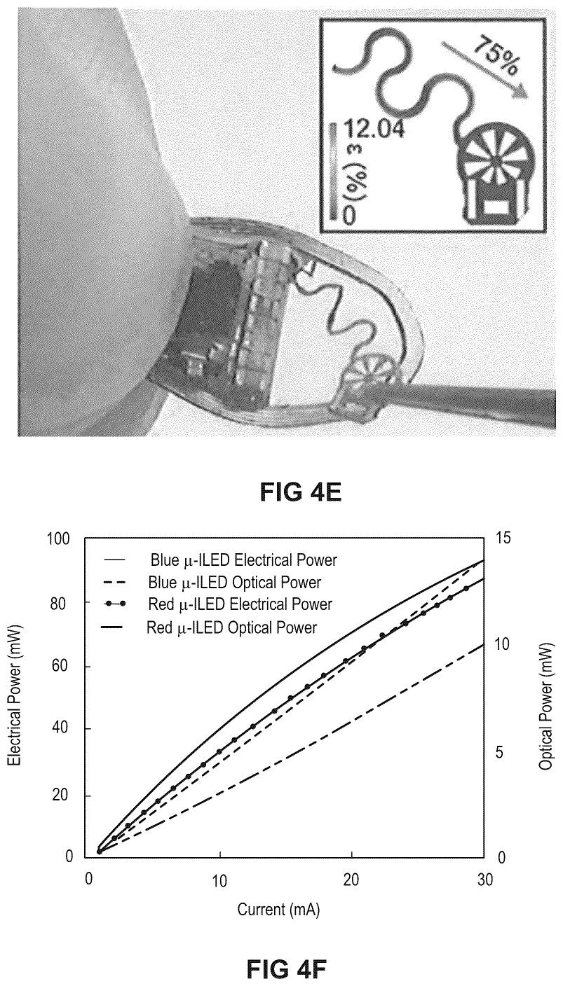

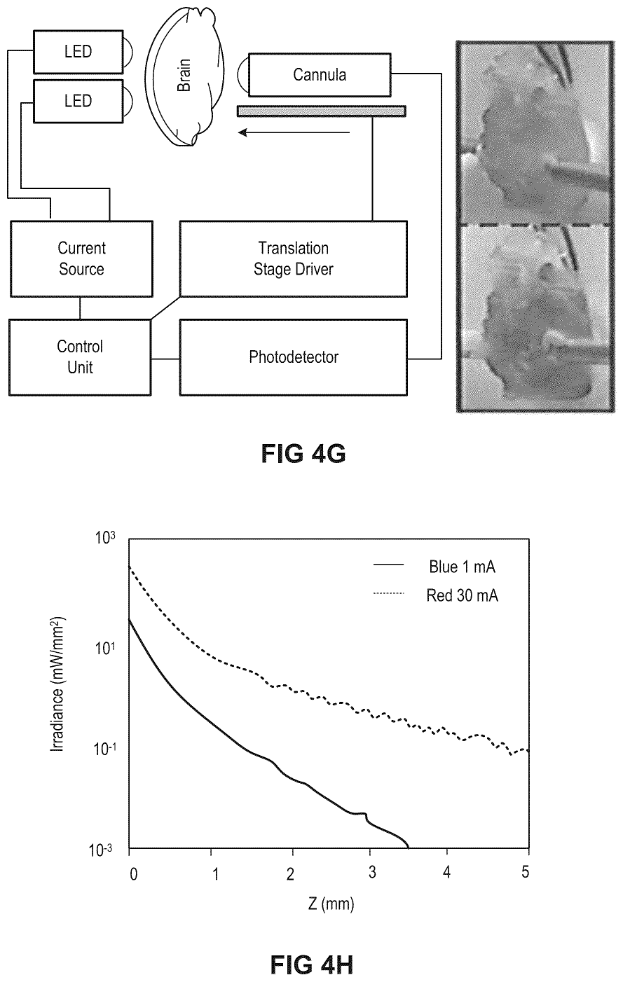

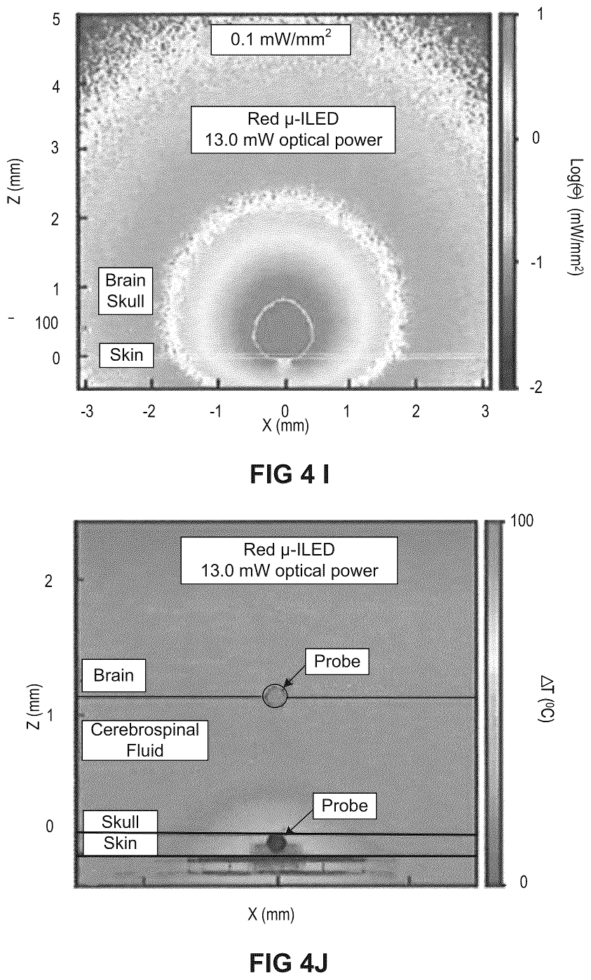

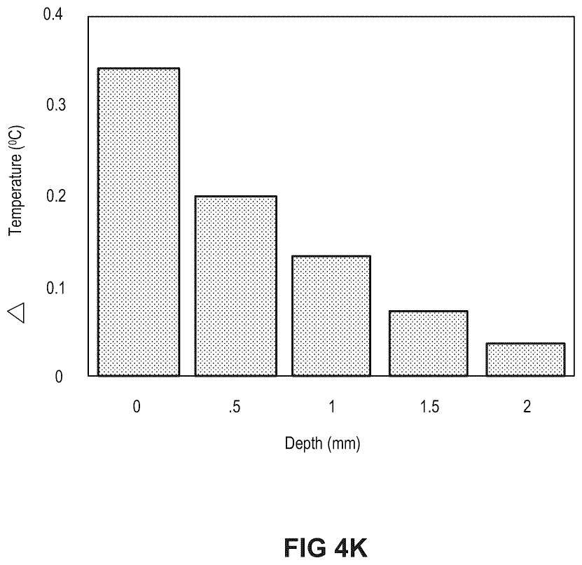

FIG. 4 illustrates transcranial device operational principles and characterization according to one example embodiment. FIG. 4A outlines the operational parameter space for the devices described in embodiments herein. In this stimulation regime, very high light intensities can penetrate the skull and reach areas of interest in the brain while keeping the blood-brain barrier intact. An example of typical parameter choices to enable transcranial stimulation is a duty cycle of 1% (10 Hz, 1 ms pulses) and a high optical power of 13 mW that can be sustained with electrical power harvesting at a rate of 0.71 mW. Thus, according to the teachings of the present disclosure, optical power demands may be balanced by a lowered duty cycle, enabling high energy events even when wireless power harvesting is low.

The electrical makeup shown in FIG. 4B illustrates device components and antenna designs that reduce the size of the device and enable the delivery of high intensity light pulses resulting in a weight of 76 mg, with dimensions of 11.73 mm×7.95 mm×0.39 mm. Specifically, the current limiting resistor, which determines light intensity, is drastically reduced (1 Ω±0.05 SI) to enable currents up to 30 mA, limited by the internal resistance of the μC (30Ω) in current sink configuration. Furthermore, LDO voltage (3.3 V) is chosen to enable increased current delivery for the red μ-ILED's (2.2 V at 30 mA) to produce high powered pulses for transcranial optogenetic stimulus.

FIG. 4C shows an exploded view schematic of the transcranial device. Positioning of the capacitor bank to the front of the device to enable a flush mounting of the system with the skull is critical for the implantation process. A photographic image of the device balanced on a finger is shown in FIG. 4D, highlighting the miniaturized form factor resulting in a displacement of 17.9 mm³. The star shaped μ-ILED holder has been engineered to enable facile surgical application to the skull. The tab, reinforced with copper strips that plastically deform, keeps the shape when bent to a 90 degree angle prior to the surgery, for mounting in a stereotactic setup. The star shaped flexible substrate further enables the precise alignment of the device with the target due to increased visibility of markers on the skull and offers a large surface area for UV crosslinked glue. Surgical procedures are described in greater detail in the Methods section below.

To enable flexible placement within the device perimeter, serpentine interconnects are engineered to accommodate movement as shown in the photographic image of FIG. 4E. The inset displays a finite element simulation with 75% strain applied (2.05 mm displacement) which results in a strain of 3.61% in the copper layer. This strain level is below plastic deformation, enabling repeated placement of the probe without impact on performance.

Optical output power of the device for two different μ-ILED wavelength is characterized in FIG. 4F. Electrical power input and resulting optical power is measured using an integration sphere, as detailed in the Methods section below. The 11-ILED can provide a maximum optical power of 14 and 13 mW for blue and red μ-ILED's, respectively, with a maximum current of 30 mA, resulting in a power consumption of 92 and 67 mW, respectively. This results in an efficiency of 19.4% for red and 11.4% for blue at 30 mA. The increased efficiency and longer wavelength of red light, which is less prone to absorption by tissue and bones, makes red light appropriate for transcranial stimulation of deep brain regions.

A customized apparatus may be used to measure light propagation in the intact skull and brain of mice, as illustrated in FIG. 4G. The optical power measured along the normal plane from the μ-ILEDs for different input optical powers is plotted in FIG. 4H for both red and blue μ-ILEDs. These results show that 13.05 mW of optical power (226.56 mW/mm² irradiance at the μ-ILED's surface) penetrates 5 mm through the skull and brain with an irradiance three order of magnitude lower (0.22 mW/mm²) for red light μ-ILEDs. This result is above the illumination threshold of red shifted opsins.

These experimental results form the basis for a Monte Carlo simulation to predict penetration of red light through the skull. A multilayer domain is implemented to consider a skull thinned to 50 μm, the skin over the skull, and the brain tissue (gray matter). The photon fluence, equivalent to the irradiance of illumination, normalized with respect to the input optical power for the red (13.05 mW, as illustrated in FIG. 4I) and blue μ-ILEDs predicts illumination volume on the order of 87.07 and 1.54 mm³, respectively. In addition, the transcranial propagation depth into the brain tissue, at a reference irradiance threshold of 0.1 mW/mm², is expected to reach 4.8 mm for red and 1.4 mm for blue illumination.

There is a direct correlation of light propagation, estimated using Monte Carlo simulation, and that obtained experimentally for both red and blue μ-ILEDs. These illumination parameters are appealing for numerous scenarios of optogenetic neuronal manipulation, especially for activation of large cortical areas or deeper regions of the brain, such as hippocampus, thalamus, and even hypothalamus and hindbrain. The predictions made using Monte Carlo simulations with subsequent experimental validation yield a powerful strategy to predict outcomes of illumination, especially in the context of parameters provided by the transcranial devices described herein. These illumination parameters are appealing for numerous scenarios of optogenetic neuronal manipulation, especially for the activation of larger areas and deeper subcortical regions of the brain.

Steady state finite element simulations in FIG. 4J show the thermal impact of high intensity pulses on physiology. The thermal impact of the light source is simulated with a 54.1 mW thermal power input, a value derived from the previously measured electrical input power and efficiency of the μ-ILED, which is represented by a 0.24×0.24×0.1 mm thermal actuator site. The thermal power is representative of the maximum operational conditions the wireless and battery free electrical system is capable of, which is created by 13 mW optical input power at the μ-ILED location with 10 Hz frequency and 1% duty cycle. In this example, a starting temperature of 36° C. is selected without considering perfusion of the tissue resulting in a worst case scenario, as temperatures in vivo are expected to be lower due to cerebrospinal fluid (CSF) perfusion. From the simulation it is evident that heat preferentially spreads towards the skin of the scalp, which has a relatively high thermal conductivity of 0.56 W m⁻¹ K⁻¹, and only a fraction of the heat penetrates the skull and enters the CSF and brain surface. Thermal impact on the skull in this condition never exceeds 0.90° C., and thermal impact on the brain tissue never exceeds 0.37° C. Experimental measurements of the thermal impact are validated by using an intact mouse skull ex vivo with a device external to the skull. The results indicate a strong match to predicted temperature from finite element modeling as shown in FIG. 4K.

Finite element simulations for a thicker skull (0.25 mm, representing a skull without surgical alteration) show a steady state temperature of 0.25° C. at the CSF/Brain interface, a 32% reduction in thermal impact compared to a thinned skull. Optical penetration depth is not significantly affected for red-shifted opsins by maintaining a thicker skull. In this example, illumination volume in comparison to a thinned skull is reduced by ˜20% for red light and 60% for blue light. Transient simulations performed in the thinned skull model at two indicated probes located at the skull/LED and Brain/CSF interfaces are used to record the transient temperature change over a five second period for a μ-ILED operating at 10 Hz and 1% duty cycle. Results of these investigations are shown in FIG. 4K. In this example, a short term temperature increase of 2.55° C. takes place at the skull/LED interface with an average increase of 0.247° C. after 5 seconds. Temperatures at the brain/CSF interface show no pulsatile component. Due to the low increases in temperature in a worst-case scenario that does not include perfusion cooling, and expect thermally driven changes in neuronal activity are not expected.

The circuit diagrams illustrated in FIGS. 1, 2 and 4 are described above in the context of using the capacitor elements (capacitive bank) as a replacement for a battery. In other embodiments, a battery may be used in conjunction with (or in place of) the capacitive elements. For example, a battery may be selected to provide an initial power bias and the remaining power may be provided by the capacitive elements as described above. Such an embodiment may enable, for example, increased operational arena size and/or increased power when needed. The battery may include any known implantable battery of a size consistent with the overall dimensions of the implantable device described herein. In addition, such a battery may include a rechargeable battery, and the controller may charge the battery using any “excess” power available in a given operating arena.

Transcranial Optogenetic Stimulation of Secondary Motor Cortex in Freely Moving Mice

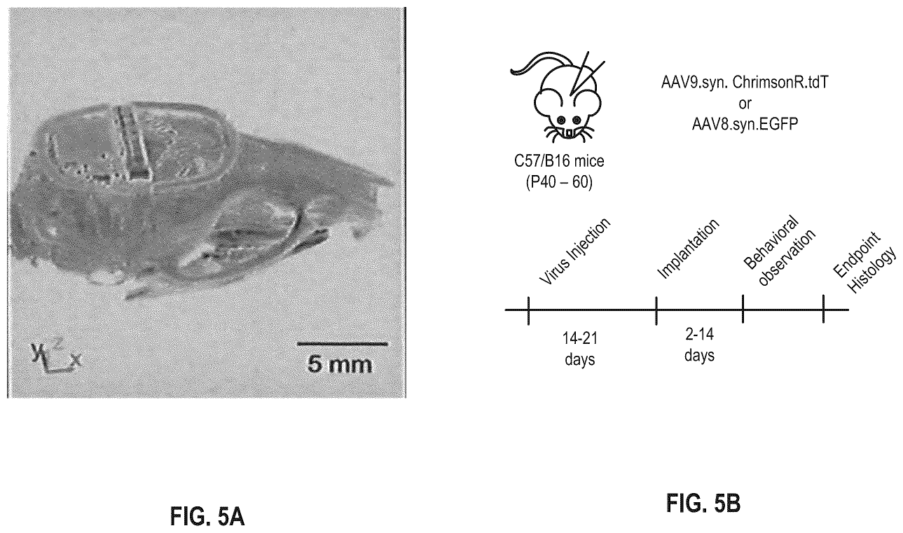

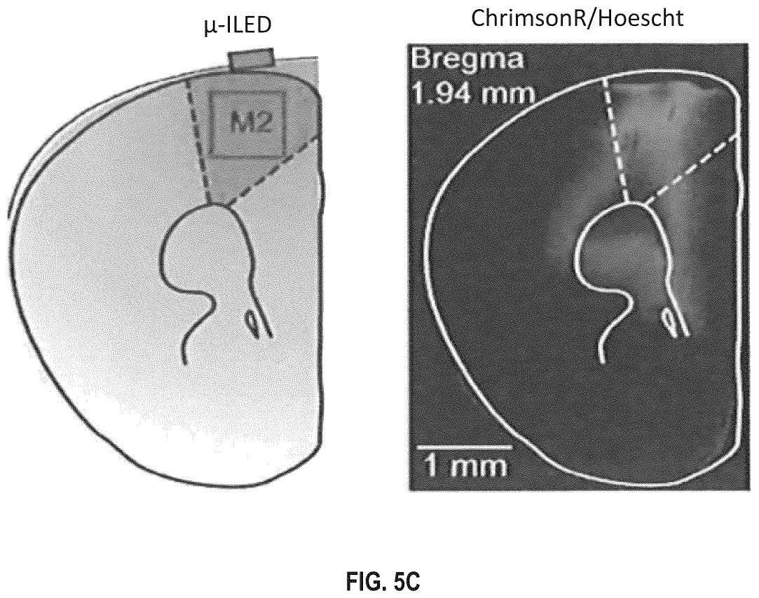

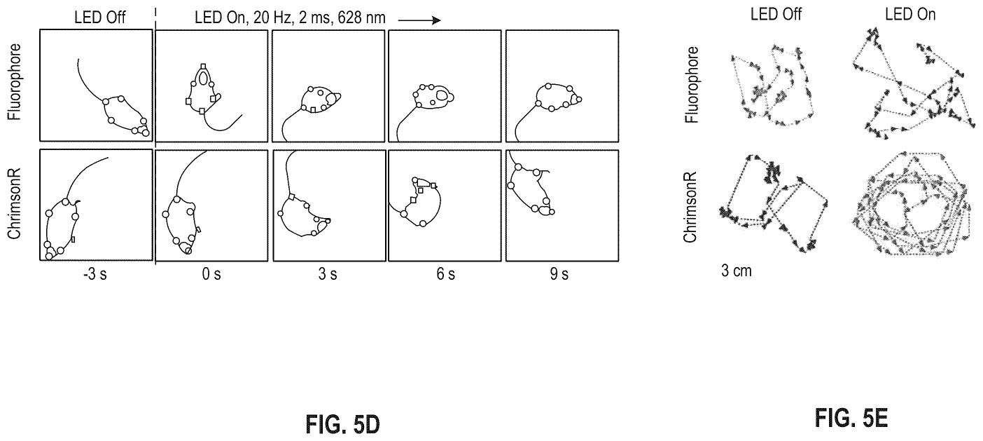

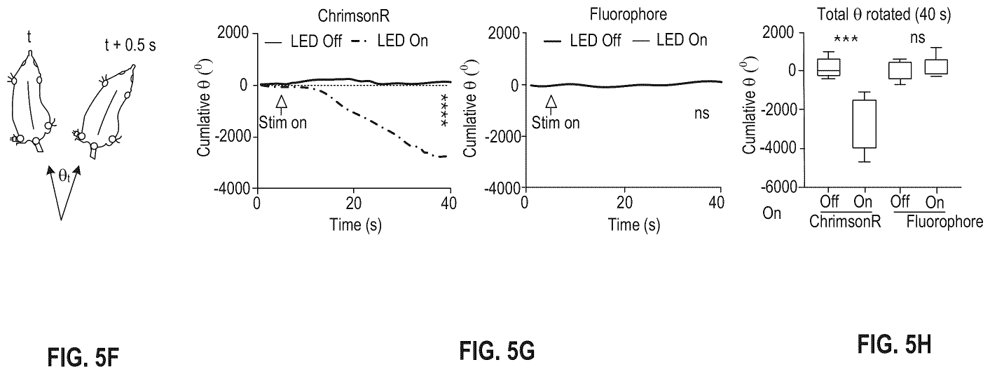

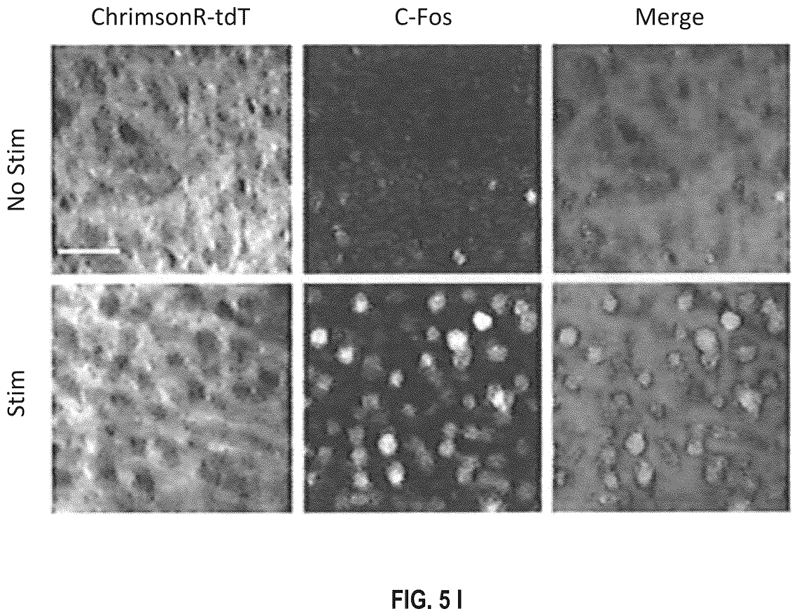

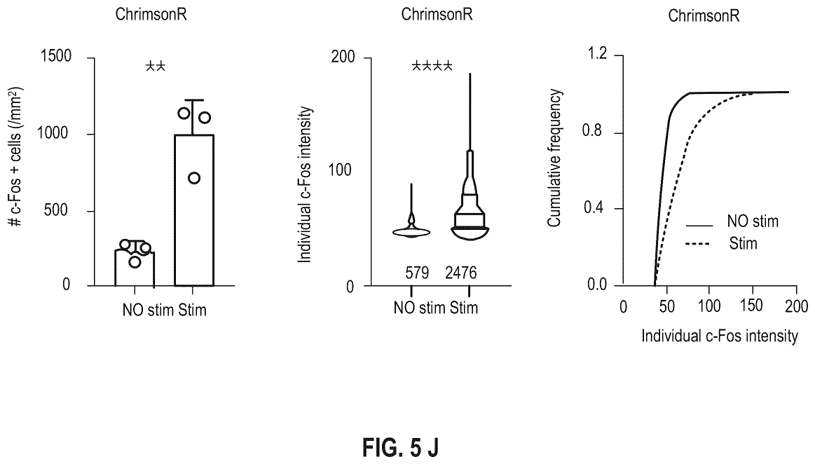

To demonstrate the feasibility of transcranial stimulation using wireless optogenetic devices with capacitive power storage in freely behaving mice, we target the secondary motor cortex (M2) that produces robust motor behavior upon activation. A CT image of the subdermally implanted device is shown in FIG. 5A. Adult mice are injected with an adeno-associated virus encoding the red light-sensitive channelrho-dopsin ChrimsonR (AAV9-syn-ChrimsonR-tdT) or EGFP (AAV8-syn-EGFP) in the right M2 (FIGS. 5B and 5C). Implant devices according to the teachings herein are implanted 2 to 3 weeks later following a standard procedure, with optional skull thinning over the site of injection to maximize light penetration. Animals are allowed to recover for at least 2 days before behavioral experiments. The devices are programmed to provide tonic high frequency stimulation at 20 Hz (2 ms pulse width, 628 nm). Experimental data demonstrate that unilateral wireless transcranial optogenetic stimulation in M2 produces robust rotational behaviors in mice expressing ChrimsonR, while mice expressing fluorophore control do not show significant rotational behavior during stimulation (FIGS. 5D-5H). These results confirm that the devices substantially activate opsins in cortical regions through the skull. The activation is unlikely to be caused by nonspecific thermal effects since mice without opsin expression do not display significant behavioral changes. To further confirm the activation of motor neurons, a subgroup of mice expressing ChrimsonR received light stimulation before tissue halvesting to induce neuronal activity-driving immediate early gene expression (c-Fos). The number of c-Fos-positive cells and individual c-Fos intensities are significantly higher in the M2 of mice that received stimulation comparing to no-stimulation controls (as shown in FIGS. 5I and 5J). Altogether, these results demonstrate clear optogenetic effects on motor behavior and neuronal activity, supporting the capability of high-intensity operation of the presented wireless platfom1 to achieve transcranial activation.

### SUMMARY

The wireless battery free and fully implantable optogenetic activation devices presented herein utilize miniaturized, digitally managed capacitive energy storage to harvest otherwise lost energy. This approach provides the ability to power events that exceed harvesting capabilities of conventional optogenetic stimulation devices to enable two applications—operation in large arenas and transcranial optogenetic stimulation. This approach more than quadruples usable arena volumes from 13,500 to 68,600 cm³ (3), with a single antenna design, and it can be scaled to very large arenas with multiple antenna and RF supplies. The capability to deliver energy in high powered pulses increases light output capability of wireless, battery-free devices by 1175% over previous reports, enabling wireless transcranial stimulation previously only achieved in head fixed and anesthetized animals. Ex vivo measurements and Monte Carlo simulations reveal that penetration in deep brain region with sufficient power to activate widely available red shifted opsins and reach nearly all areas of the mouse brain with most recent optogenetic tools. As demonstrated above, high-powered light pulses from the wireless, battery-free subdermal implanted devices can transcranially stimulate the motor cortex to evoke motor responses.

Collectively, the devices described herein significantly expand experimental paradigms for neural activity control in freely moving subjects in ethologically relevant environments and with minimally invasive transcranial optogenetics. Fundamentally, the strategies introduced and demonstrated here for small rodent subjects also apply to other animal models, including 3D navigating species and large animals. Further, the energy delivery and management strategies may provide the foundation to advance fidelity of other techniques that have recently been shown in wireless and battery free device formats, including photometry, phototherapy, as well as electrical stimulation for cardiac and neural stimulation.

Material and Methods

Device Fabrication

Pyralux AP8535R served as the substrate for the flex circuit. Direct laser ablation (LPKF U4) was used to structure the top and bottom copper layers (17.5 μm) on substrate polyimide layer (75 μm). Ultrasonic cleaning (Vevor; Commercial Ultrasonic Cleaner) was carried out with flux (Superior Flux and Manufacturing Company; Superior #71, 10 minutes) followed by isopropyl alcohol (MG Chemicals, 2 minutes) wash. Devices were rinsed with DI water to remove any remaining particles. Copper wire (100 μm) and low temperature solder (Chip Quik; TS391LT) were used for via connections. After assembly device components were fixed in place with UV-curable glue (Damn Good 20910DGFL) followed by curing with UV lamp (24 W) for 5 minutes. Devices were encapsulated with parylene coating via chemical vapor deposition (CVD).

Electronic Components

Electrical components are generally selected to provide a minimal device footprint, outline, and volume. Low temperature solder (Chip Quik; TS391LT) was used for manual soldering of components onto device. A rectifier composed of two shottky diodes (40 V, 30 mA, MCC RB751S-40DP) and a tuning capacitor of 82 μF (TDK, CGA2B3×7R1H104K050BE) and a 2.2 μF smoothing capacitor (Samsung CL03A225MQ3CRNC) were used for the rectifier. Eight 22 μF capacitors (Samsung CL05A226MQ5N6J8) were used to create a capacitor bank with a total capacitance of 176 μF. A Zener diode (5.6 V, 100 mW, Comchip CZRZ5V6B-HF) was used for overvoltage protection. A low-dropout regulator with fixed internal output (2.7 V, Fairchild FAN25800, long-range applications, 3.3 V; ON Semiconductor NCP163AFCT330T2G, transcranial applications) managed voltage to the implants. A small outline μC (ATTiny 84A 3 mm×3 mm; Atmel) with wide operational voltage capabilities was used to control μ-ILED activation. The μC firmware was programmed to power μ-ILED with energy stored in the capacitor bank by sinking current through the μ-ILED at relevant time points. The blue μ-ILED (CREE TR2227) current was limited by a current limiting resistor (150Ω for low load applications, 1Ω for high load applications) to control irradiance. The red μ-ILED (Epistar ES-AEHRAX10) current was limited by the copper trace of the device (1Ω) to control irradiance for high load applications.

Experimental Animals/Surgical Procedure

All procedures were performed in accordance with protocols approved by the IACUC at Northwestern University. Right primary motor cortex (M1) was targeted for stereotactic injection in 6-8 week-old C57BL6 mice (Jackson; JAX000664) under isoflurane anesthesia using the following coordinates relative to bregma: x=1.5, y=0.5, z=0.6. Viruses (AAV9-syn-ChrimsonR-tdT, addgene #59171; AAV8-syn-EGFP, UNC vector core) were diluted to a titer of ˜5×10¹² vg/ml in PBS and injected using a glass pipette and a micro-injector. After allowing at least 21 days for virus expression, the skull was thinned to ˜50 μm over the previous injection site and the LED was attached to the skull using a drop of UV-cured optical glue (Norland). The body of the device, which was sterilized in alcohol prior to implantation, was then secured to the skull using dental cement or Vetbond (3M) and the skin was sutured over the device. Animals recovered for 48-72 hours before the behavior experiment.

Behavioral Experiments

Animals were placed in a 15×15-cm arena equipped with an antenna connected to a Neurolux system. After a 2-min-long acclimatization period, animals were recorded for 3 min with the antenna turned off and then another 3 min with the antenna powered on. The devices were configured to deliver 2-ms long pulses at 20 Hz. Videos were recorded using a Raspberry Pi camera with a resolution of 1,280×720 at 25 fps. The snout, ears, hind legs, and the base of tail of each mouse were labeled using Deeplabcut. For each video, one frame per second was automatically selected in a uniform manner to be manually labeled, generating the training and testing dataset for the Deeplabcut algorithm. The test error with a p-cutoff value of 0.6 was 4.85 pixels. To quantify the rotational behavior of mice, one automatically labeled frame was selected per 0.5 s. The “head” position was defined as the centroid of the coordinates of the snout and ears, while the “back” position was defined by the centroid of the coordinates of two legs and the base of the tail. The body vectors were created from “back” to “head” positions for all labeled frames. Two body vectors separated by 0.5 s were designated as v1 (x1, y1) and v2 (x2, y2), respectively, and the angle of mice rotated in this period was calculated as follows:

angle=arctan 2(x1y2−y1x2,x1x2+Y1Y2),

The angle was converted from radian to degrees. The cumulative degrees rotated during the initial 40 s were plotted for each experiment.

Immunohistochemistry

Immunostaining for c-Fos was performed on 60-μm vibratome sections by permeabilizing the tissue in 0.4% Triton-X in PBS for 15 min, blocking in PBS with 0.5% Triton and 10% bovine serum albumin for 1 h, performing the primary incubation in 1:5,000 rabbit anti--<:-Fos (Synaptic Systems, category no. 226 003, RRID: AB_2231974) in PBS overnight at 4° C., washing three times in PBS, performing the secondary incubation in 1:500 AlexaFluor647-conjugated donkey anti-rabbit (Thermo Fisher Scientific, category no. A·31573, RRID: AB_2536183), and washing three times in PBS as described in previous publications (50-53). After staining, the slices were mounted in a 9:1 mixture of glycerol and PBS containing Hoechst 33342 (2.5 μg/mL, Thermo Fisher Scientific). For c-Fos quantification, coronal brain sections were imaged using an Olympus VS120 microscope. All imaging parameters were constant across all samples, and each channel was imaged sequentially with a 10× objective. Two regions of interest (ROls) of M2 (648×648 μm2) per animal, selected from two different coronal sections, were used for analysis. Analysis was carried out in FIJI (54) using auto thresholding and particle analysis scripts. The same analysis parameters were applied across all ROls.

Device Characterization

Power consumption of the device was characterized by measuring current into the μC and μ-ILED with a current probe (Current Ranger LowPowerLab) at the defined system voltage. Voltage of the capacitor bank was measured with an oscilloscope (Siglent SDS 1202X-E). Siglent SSA 3032X Spectrum Analyzer was used to verify the resonant frequency of the secondary antenna at 13.56 MHz. Power harvesting of devices with 6, 8, and 10 antenna turns were characterized in the 70×70 cm cage by placing each device on a 3 cm mount at the center of the field and measuring the voltage output using a DMM (AN8008) with increasing loads added across the device. Arena power mapping was performed by placing 10 turn devices at relative heights of 3, 6, and 8 cm from the cage floor and measuring voltage output with a DMM at a load of 10 kΩ throughout the arena with 10 cm distance between each measurement point.

Wireless Programming.

Implants described herein were programmed using a laptop computer with a software interface to select stimulation parameters and power amplifier settings. Ex-ternal hardware included an RF amplifier (Neurolux, Inc.), tuner hardware (Neurolux, Inc.) and a custom TTL controller based on an Arduino Nano microcontroller (code available on GitHub). Wireless power to the implant was modulated with an ON/OFF keying protocol sequence that was demodulated at the implant. The protocol utilized 12 bits of data to select up to 64 duty cycles and 64 frequencies, resulting in a programming time of 20 s, Programming was experimentally validated by μ-ILED output measurements using a custom photodetector and transimpedance amplifier setup

Electromagnetic Simulation

The commercial software ANSYS HFSS was used to perform electromagnetic finite element analysis to determine the magnetic field distribution inside a 70 cm×70 cm×15 cm cage (length×width×height) enclosed by a copper wire antenna (diameter=3 mm) with two loops. The bottom and top loops were placed at 4 cm and 11 cm, respectively, above the cage floor to create a uniform magnetic field. A lumped port was used to obtain the port impedance Z of the wire antenna and tune it to a working frequency of 13.56 MHz. An adaptive mesh (tetrahedron elements) and a spherical radiation boundary (radius of 5000 mm) was adopted to ensure computational accuracy. The bulk conductivity, relative permittivity, and relative permeability of copper were σ=5.8×10⁷ S/m, ε=1, and μ=0.99, respectively.

Optical Characterization

The optical characterization was performed using an integrating sphere (OceanOptics FOIS-1) with a factory calibrated light source (OceanOptics HL-3 plus). The μ-ILEDs were sourced with a current source (Keitheley 2231A-30-3, Tektronix) to provide precise current to the device. The μ-ILEDs were mounted on a heat sink substrate to dissipate the excessive heat that could damage the μ-ILEDs. After light source calibration the total irradiance flux spectra was collected using the vendor's provided software (OceanView), which contains the spectral power density (W/nm) of the light emitted by the μ-ILEDs. This spectra collection was repeated for each test driving current. The total power was then calculated by integration of the irradiance flux using a customized MATLAB script using an integration windows of 400-600 nm for blue μ-ILED and 550-700 nm for red μ-ILED.

Optical Simulations

The optical simulation of transcranial optogenetic stimulation was implemented using the Monte Carlo method. The volume of the numerical simulations was comprised of 850 bins, each with (8 μm), which represents a total volume of (6.8 mm). The blue (460 nm) illumination source was a rectangular emitting surface of 0.22×0.27 mm², whereas the red (628 nm) illumination source is 0.24×0.24 mm², both with a 120° emission angle. In the simulation 6.1×106 photons were launched. The material's optical properties (absorption, scattering coefficients and dissymmetry factor: μa, μs, g) for the simulations were brain tissue (both gray and white matter), skull (50 μm), skin (700 μm), and air. An iterative comparison of optical parameters for brain tissue with measured experimental data revealed optical parameters values that stay within the margin of those reported in the literature (blue: μa=1.5 cm−1, μs=300 cm−1, and g=0.83; red: μa=0.6 cm−1, μs=75 cm−1, and g=0.83), which were the ones used in the data analysis. Once the simulation for each wavelength was performed, postprocessing of the photon flux provided the illumination profiles, illumination volume, and irradiance decay normal to the μ-ILED's plane. The dimensional rendering for transcranial illumination with red light producing 5 mW of optical power was generated using Paraview 5.7.0.

Characterization of Transcranial Light Propagation

The Transcranial Light Propagation Characterization in Fresh Mice Brain Tissue was Carried out using a homemade optical characterization apparatus, see FIG. 4g. This system was comprised of a 10 mm long 0.22 numerical aperture (NA) optical fiber cannula (0.2 mm core diameter) coupled to a high sensitivity photomultiplier (Model 2151, New Focus Inc.) using a fiber patch cable, both mounted on a one-dimensional linear translation stage (KMTS25E, Thorlabs Inc.). A customized MATLAB script was implemented to synchronize the current source to drive the 11-LEDs (Keitheley 2231A-30-3, Tektronix), the data acquisition system (U3-HV, LabJack Corporation) to record the photovoltage generated by the photomultiplier, and the translation stage controller (KDC101, Thorlabs Inc.). The red and blue μ-ILEDs were mounted side by side on a probe to allow simultaneous characterization, see FIG. 4h. At each 100 μm step, five test currents were probed simultaneously for blue illumination (1, 2, 3, 4, and 5 mA) and four for red illumination (1, 10, 20, and 30 mA). Mice heads were prepared the same day within one hour prior to the characterization. They had the brain intact, thinned skull at the location of the illumination site and a 1 mm hole on the opposite side for fiber insertion. Data was acquired as the cannula was inserted into the fresh and unperturbed brain tissue to avoid artificial scattering by airgaps produced while retracting the fiber. The comparison of the simulation illumination power as a function of depth consisted in integrating an area equal to the core of the optical fiber (200 μm). Due to the distribution of the μ-ILED's on the probe, the area of integration is not at the center of either μ-ILED, but in between.

Thermal Finite-Element Analysis Model

Ansys® 2019 R2 Steady-State Thermal and Transient-State Thermal was utilized for static-thermal and transient-thermal finite-element modeling. The models were used to study the changes in temperature of the skull and brain tissue surrounding the μ-ILED. Components of the transcranial device, including the copper, PI, and parylene encapsulation layers, were simulated in accurate layouts and with exact topologies within an octagonal area extending at least 0.95 mm in each direction from the μ-ILED. Program Controlled Mechanical Elements were used to simulate each model. The resolution of the mesh elements was set to 4 and a minimum edge length of 6.5305 μm was used. Mesh convergence was ensured by using at least two elements in each direction. The thermal conductivity, heat capacity, and mass density of the materials used in the simulations was 130 W m⁻¹ K⁻¹, 490 J kg⁻¹ K⁻¹, and 8920 kg m⁻³ for the μ-ILED; 0.57 W m⁻¹ K⁻¹, 4068 J kg⁻¹ K⁻¹, and 1017 kg m⁻³ for the cerebrospinal fluid; 400 W m⁻¹ K⁻¹, 385 J kg⁻¹ K⁻¹, and 8933 kg m⁻³ for the copper traces; 0.37 W m⁻¹ K⁻¹, 3391 J kg⁻¹ K⁻¹, and 1109 kg m⁻³ for the tissue; 0.126 W m⁻¹ K⁻¹, 837 J kg⁻¹ K⁻¹, and 1110 kg m⁻³ for the parylene encapsulation; and 0.12 W m⁻¹ K⁻¹, 1090 J kg⁻¹ K⁻¹, and 1420 kg m³ for the PI.

Characterization of Transcranial Thermal Propagation.

The transcranial thermal impact characterization in mouse brain tissue ex vivo was carried out using a custom-built temperature probe and positioning stage. A source meter unit was used to measure signals from an 0201 negative-temperature coefficient sensor mounted on a 8.3-mm-long, 0.5-mm and 0.1-mm-thick custom probe to acquire the tissue temperature with millikelvin resolution. After calibration of the temperature sensor, the transcranial stimulation device was attached to the skull with surgical glue, analogous to the procedure described in Experimental Animals/Surgical Procedure. A one-dimensional positioning stage was used to insert the temperature senor probe through a 1 mm opening opposite the illumination site. Temperature measurements were taken at 0.5-mm increments starting from the skull at the illumination site. For each measurement, the device was operated at 10 Hz and 1% duty cycle for 5 min. Afterward, the temperature of the tissue was reset to ambient temperature bya cooldown period of 2 h.

Mechanical Simulations

Ansys® 2019 R2 Static Structural was utilized for static-structural Finite Element Analysis (FEA) simulations to study the elastic strain in the transcranial device and long-range device when the tethers were stretched. The components of the devices, including the copper traces, PI, μ-ILED, and parylene encapsulation layers, were simulated in accurate layouts and using their exact topologies. The model was simulated using Program Controlled Nonlinear Mechanical Elements, with an element size manually input as 2E-2 mm. The Young's Modulus (E) and Poisson's Ratio (v) are EPI=4 GPa, vPI=0.34; ECU=121 GPa, vCU=0.34; EParylene=2.7579 GPa; vParylene=0.4; Eμ-ILED=343 GPa, vμ-ILED=0.28. For each device, a fixed support was added to the respective face. The applied strains for the transcranial device and long-range device serpentines were applied using a displacement as the load on the faces in the direction of the arrow shown in FIGS. 2E and 4E, and were 75% (0.65 mm) for the transcranial device and 110% (˜5.53 mm) for the long-range device.

MicroCT/MRI Imaging

Mice were placed in an induction chamber and anesthetized with 3% isoflurane in oxygen. Mice were then transferred to an imaging bed with isoflurane delivered via nosecone (1-2%). A separate mouse bed was used for both imaging systems. After being placed in the prone position, the head of the mouse was immobilized with ear and tooth bars. Respiratory signals were continuously monitored during μCT imaging with a digital system (Mediso-USA, Boston, MA). A preclinical microPET/CT imaging system (Mediso nanoScan scanner) was used to acquire images of the mice. Data was acquired with 2.17 magnification, 33 μm focal spot, 1×1 binning, using 720 projections over a full circle, an exposure time of 300 ms, and a peak tube voltage of 70 kV. A butterworth filter backprojection software (Mediso) was used to reconstruct each projection with a voxel size of 34 μm. A 9.4 T Bruker Bio spec MRI system with a 30 cm bore and a 12 cm gradient insert (Bruker Biospin Inc, Billerica, MA) was used to take MRI images. A MR-compatible system (SA Instruments, Stonybrook, NY) was used to continuously monitor respiratory signals, warm circuiting pads were used to maintain the body temperature of the animals. The animal bed was placed inside a 72 mm quadrature volume coil in transmit-only mode (Bruker Biospin, Inc, Billerica, MA). The actively decoupled 4-channel receiver coil (Bruker Biospin, Inc, Billerica, MA) was mounted to the animal bed. Accelerated spin echo sequences (Turbo Rapid Acquisition with Relaxation Enhancement, TurboRARE) were used to collect images that were oriented in the axial, sagittal, and coronal configurations. The following specifications were used for each MRI scan: TR/TE=1250 ms/21.3 ms, MTX=256×256, FOV 3×3 cm, RARE factor 8, 7-13 slices of 0.75-1 mm thick, flip back enabled, 3 signal averages and a total acquisition time of ˜2 mins per scan. Data was visualized and reconstructed with Amira 6.7 (FEI, Houston, TX) so that both MRI and μCT images were able to be manually superimposed over one another. Image artifacts in the CT images were further processed and reduced using Amira with non-local means filtering.

The foregoing description of animal models using the optogenetic stimulation device described herein is provided as an examples of in-vivo utilization of the present disclosure. In other embodiments, the optogenetic stimulation devices described herein can be used as transcranial and/or subdermal therapeutic and/or diagnostic devices in human subjects.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

“Circuit” and “Circuitry”, as used in any embodiment herein, may comprise, for example, singly or in any combination, hardwired circuitry, programmable circuitry such as processors comprising one or more individual instruction processing cores, state machine circuitry, and/or firmware that stores instructions executed by programmable circuitry, hardware embodiments of accelerators such as neural net processors and non-silicon implementations of the above. The circuitry may, collectively or individually, be embodied as circuitry that forms part of a larger system, for example, an integrated circuit (IC), system on-chip (SoC), application-specific integrated circuit (ASIC), programmable logic devices (PLD), digital signal processors (DSP), field programmable gate array (FPGA), logic gates, registers, semiconductor device, chips, microchips, chip sets, etc.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents. Various features, aspects, and embodiments have been described herein. The features, aspects, and embodiments are susceptible to combination with one another as well as to variation and modification, as will be understood by those having skill in the art. The present disclosure should, therefore, be considered to encompass such combinations, variations, and modifications.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

What is claimed:

1. A subdermally implantable optogenetic device, comprising:
a subdermally implantable housing; and
an optogenetic stimulation circuit disposed within the subdermally implantable housing, the optogenetic circuit comprising:
energy harvesting circuitry to receive radio frequency (RF) energy;
one or more capacitor storage elements to store energy associated with the RF energy;
a light emitting diode (LED) to generate a light source at a selected frequency; and
controller circuitry to discharge the one or more capacitor storage elements at a selected duty cycle by coupling the one or more capacitive storage elements to the LED to cause the LED to generate pulsed light at the selected duty cycle; the controller circuitry also to charge the one or more capacitive elements by coupling the one or more capacitive storage elements to the energy harvesting circuitry.

2. The subdermally implantable optogenetic device of claim 1, wherein the energy harvesting circuitry comprising an antenna to receive the RF energy.

3. The subdermally implantable optogenetic device of claim 2, wherein the antenna comprising a coil having a selected number of turns to generate a desired power from the RF energy.

4. The subdermally implantable optogenetic device of claim 1, wherein the one or more capacitor storage elements having a capacitance value to charge to a selected voltage level, the capacitance value also to have a charge time that charges the capacitor storage elements at a rate that is faster than the selected duty cycle for optogenetic stimulation.

5. The subdermally implantable optogenetic device of claim 1, wherein the controller to operate in a high power mode and a low power mode, wherein the high power mode to discharge the one or more capacitor storage elements faster than in the low power mode.

6. The subdermally implantable optogenetic device of claim 1, wherein the one or more capacitive storage elements includes a plurality of ceramic capacitor elements coupled in parallel.

7. The subdermally implantable optogenetic device of claim 1, wherein the housing is formed of parylene.

8. The subdermally implantable optogenetic device of claim 1, wherein the LED is micro-inorganic LED (u-ILED).

9. The subdermally implantable optogenetic device of claim 1, wherein the power harvesting circuitry comprises a linear drop out regulator (LDO) coupled to the capacitor storage elements and the LED to deliver substantially consistent voltage levels to the LED.

10. The subdermally implantable optogenetic device of claim 1, further comprising a battery, wherein the controller circuitry further to control the battery and the capacitive storage elements to deliver power to the LED from both the battery and the capacitive storage elements.

11. A transcranial implantable optogenetic device, comprising:
a transcranial implantable housing; and
an optogenetic stimulation circuit disposed within the transcranial implantable housing, the optogenetic circuit comprising:
energy harvesting circuitry to receive radio frequency (RF) energy;
one or more capacitor storage elements to store energy associated with the RF energy; and
a light emitting diode (LED) to generate a light source at a selected frequency;
controller circuitry to discharge the one or more capacitor storage elements at a selected duty cycle by coupling the one or more capacitive storage elements to the LED to cause the LED to generate pulsed light at the selected duty cycle; the controller circuitry also to charge the one or more capacitive elements by coupling the one or more capacitive storage elements to the energy harvesting circuitry; wherein the controller to operate in a high power mode and a low power mode, wherein the high power mode to discharge the one or more capacitor storage elements faster than in the low power mode.

12. The transcranial implantable optogenetic device of claim 11, wherein the energy harvesting circuitry comprising an antenna to receive the RF energy.

13. The transcranial implantable optogenetic device of claim 12, wherein the antenna comprising a coil having a selected number of turns to generate a desired power from the RF energy.

14. The transcranial implantable optogenetic device of claim 11, wherein the one or more capacitor storage elements having a capacitance value to charge to a selected voltage level, the capacitance value also to have a charge time that charges the capacitor storage elements at a rate that is faster than the selected duty cycle.

15. The transcranial implantable optogenetic device of claim 11, wherein the one or more capacitive storage elements includes a plurality of ceramic capacitor elements coupled in parallel.

16. The transcranial implantable optogenetic device of claim 11, wherein the housing is formed of parylene.

17. The transcranial implantable optogenetic device of claim 11, wherein the power harvesting circuitry comprises a linear drop out regulator (LDO) coupled to the capacitor storage elements and the LED to deliver substantially consistent voltage levels to the LED.

18. The transcranial implantable optogenetic device of claim 11, wherein the LED is micro-inorganic LED (u-ILED).

19. The transcranial implantable optogenetic device of claim 11, further comprising a battery, wherein the controller circuitry further to control the battery and the capacitive storage elements to deliver power to the LED from both the battery and the capacitive storage elements.

20. The transcranial implantable optogenetic device of claim 11, wherein the selected duty cycle is approximately 0.1% to 30%, corresponding to a pulse rate of approximately 0.5 Hertz to 1000 Hertz; wherein wherein the controller to operate in the high power mode to discharge the one or more capacitor storage elements within 1 to 0.1 ms.; and wherein the controller to operate in the low power mode to discharge the one or more capacitor storage elements within 10 to 40 ms.
